(19) United States
(12) Patent Application Publication
(10) Pub. No.: US 2024/0041529 A1
(43) Pub. Date: Feb. 8, 2024

(54) DEVICE FOR MODELING CERVICAL ARTIFICIAL DISC BASED ON ARTIFICIAL INTELLIGENCE AND METHOD THEREOF

(71) Applicant: HealthHub Co.,Ltd., Seoul (KR)

(72) Inventors: Byung Hoon LEE, Seoul (KR); Byung II LEE, Seoul (KR); Shi Sub BYON, Seoul (KR)

(73) Assignee: HealthHub Co.,Ltd., Seoul (KR)

(21) Appl. No.: 18/258,949

(22) PCT Filed: Dec. 23, 2020
(86) PCT No.: PCT/KR20/18976

§ 371 Date: Jul. 6, 2023

(30) Foreign Application Priority Data
Dec. 23, 2020 (KR) 10-2020-0181590

(51) Int. Cl. A61B 34/10; G16H 50/50; G06T 7/33; A61F 2/30

(52) U.S. Cl. CPC A61B 34/10; G16H 50/50; G06T 7/344; A61F 2/3094; G06T 2200/08; G06T 2207/30012; G06T 2207/20081; G06T 2207/20084; A61B 2034/105

(57) ABSTRACT

The present invention relates to a device for modeling cervical artificial disc based on artificial intelligence and a method thereof, comprising generating and outputting a model of a target cervical artificial disc, by generating a learning model to estimate positions of a plurality of landmarks through training various shapes of medical images with the plurality of landmarks assigned to define an available space in which the cervical artificial disc is located, estimating the plurality of landmarks by inputting input data generated from a medical image of the available space in which the target cervical artificial disc is to be positioned into the generated learning model, generating the available space for inserting the target cervical artificial disc based on estimated landmarks, and extracting and incorporating shape information for surfaces and edges of the cervical artificial disc from the medical image in the generated available space.

### TECHNICAL FIELD

The present invention relates to a device for modeling cervical artificial disc based on artificial intelligence and a method thereof, and more particularly, provides an AI-based device and method for modeling a cervical artificial disc, comprising generating and outputting a model of a target cervical artificial disc, by generating a learning model to estimate positions of a plurality of landmarks through training various shapes of medical images with the plurality of landmarks assigned to define an available space in which the cervical artificial disc is located, estimating the plurality of landmarks by inputting input data generated from a medical image of the available space in which the target cervical artificial disc is to be positioned into the generated learning model, generating the available space for inserting the target cervical artificial disc based on estimated landmarks, and extracting and incorporating shape information for surfaces and edges of the cervical artificial disc from the medical image in the generated available space.

### BACKGROUND

A human spine is a body organ forming a center of a human body positioned in a longitudinal direction at the center of a back of the body. The spine comprises 7 cervical vertebrae (neck bone), 12 thoracic vertebrae (back bones), 5 lumbar vertebrae (lower back bones), 5 sacrum (pelvic bones), and 4 coccygeal vertebrae (tailbone), and supports a head to upper side, connects to the pelvis to lower side and transmits the body weight to a lower limb. A fiber cartilage intervertebral disc (that is, a disc) is formed between the vertebrae bodies so as to support the body and maintain the balance by combining strong ligament and muscle connected from the skull to the pelvic bone.

However, wrong posture, excessive exercise, degenerative diseases, and the like may cause diseases in the spine, and the treatment of diseases related to the spine includes an indirect treatment method through physical therapy and a direct treatment method for correcting and immobilizing the spine by mounting a separate stabilizing device on the damaged spine. That is, in cases where spine disorders are mild, physical therapy is performed, but for severe conditions affecting cervical vertebrae, thoracic vertebrae, lumbar vertebrae, sacrum, and intervertebral disc, which constitute the spine, a separate spinal stabilization device (for example, an implant such as a cervical artificial disc) should be used for treatment.

However, in the treatment using various implants, existing implants have had limitations in that a space between vertebrae cannot be completely restored, implants become obstacles that hinder the movement of the spine, transplantation is difficult during a surgical procedure, or reliability with respect to durability is deteriorated.

Therefore, the present invention provides a device and method for performing patient-customized cervical artificial disc modeling based on artificial intelligence. Specifically, a method for first generating a learning model for estimating a plurality of landmarks, and then estimating coordinates for the plurality of landmarks by inputting a medical image of a user into the learning model for estimating the landmarks and modeling the cervical artificial disc of the user by using each of the coordinates of the estimated landmarks is presented.

More specifically, in the present invention training data are generated by determining a plurality of landmarks on each collected medical images for learning purposes, and a learning model is created for landmark estimation by training the learning model with the generated training data. Next, the estimated landmarks are applied to a to a standard template for a standard artificial disc to adjust the size of the template, and the coordinates for a shape of an actual patient image with respect to a surface, a height, an edge, or a combination thereof for upper and lower plates of a disc to be operated around the center of the landmarks are acquired, and thereby a cervical artificial disc to be operated on a patient is modeled by reflecting the acquired coordinates on the surface of the size adjusted template.

Next, the prior arts existing in the technical field of the present invention will be briefly described, and then the technical aspects that the present invention aims to be distinguished from the prior arts are described as follows.

First, Korean Patent Application No. 2020-0029548 (Mar. 18, 2020) relates to a method for optimizing a design of an orthopedic component, the method for understanding the anatomical structures, both external and internal, of bones using imaging data and 3D modeling, for allowing easier design of the anatomically accurate plates, devices, and implants, in which an implant or a plate comprising at least one curved surface is created, the contour of at least one curved surface corresponds to the anatomical shape of a subject, and the anatomical shape of the subject is determined based on the image of the bone.

That is, the prior art discloses a method for enhancing the understanding of fractures around artificial implants and related anatomical structures of the forearm bone to facilitate design and selection of anatomically accurate bone plates surrounding the artificial implants.

However, according to the present invention, coordinates for a plurality of landmarks are automatically estimated from a medical image of a user by using a learning model for estimating landmarks, and a cervical artificial disc of a user is modeled using the estimated plurality of landmark coordinates, and thus the prior art and the present invention have a remarkable difference in their structural configurations.

In addition, Korean Patent Application Publication No. 2019-0140990 (Dec. 20, 2019) relates to a system and method for manufacturing a dental appliance, comprising receiving data identifying an approximate location of an individual tooth in a three-dimensional digital dental model representing an impressed position of the patient's dentition, generating a component model corresponding to the respective tooth for each of the identified approximate locations, determining a target location for the component model, generating a tooth positioning appliance design based on the determined target location for the component model, and causing a tooth positioning appliance to be fabricated based on the tooth positioning appliance design.

On the other hand, the present invention relates to cervical artificial disc modeling, and thus the application field thereof is different. In particular, the present invention uses a learning model for landmark estimation to estimate coordinates of a plurality of landmark from a medical image of a user and models a cervical artificial disc of the user by using the estimated plurality of landmarks coordinates, and thus the prior art and the present invention are different from each other in terms of technical configurations.

### BRIEF SUMMARY OF THE EMBODIMENTS

An objective of the present invention created to address the aforementioned issues, is to provide a device and a method for modeling a cervical artificial disc optimized for a user requiring cervical artificial disc replacement surgery by using artificial intelligence.

In addition, another objective of the present invention is to provide a device and a method for modeling a cervical artificial disc of a user by generating training data by determining a plurality of landmarks from a medical image for training, generating a learning model for estimating landmarks by training the learning model with the generated training data, estimating landmark coordinates either individually or collectively at once by inputting a medical image of a user into the learning model for estimating the landmarks, and using each coordinate of the estimated landmarks.

In addition, another objective of the present invention is to provide a device and a method for modeling a cervical artificial disc of a patient to be operated by adjusting a size of a standard template according to the estimated landmarks, matching the landmarks to a medical image of a patient to be operated, and reflecting shape information extracted from the matched medical image to upper and lower surfaces and edges of the template.

In addition, another objective of the present invention is to provide a device and a method capable of increasing the satisfaction of a user by increasing the success rate of a surgery by making the cervical artificial disc customized for each surgery scheduled user based on specification information comprising the height, length, width, shape, or a combination thereof of the cervical artificial disc modeled using the artificial intelligence learning model.

An artificial intelligence-based cervical artificial disc modeling device according to an embodiment of the present invention is characterized in that the device comprises: a learning model generation part configured to generate a learning model through training a plurality of landmarks constituting a plurality of spaces for a cervical disc; a landmark estimation part configured to estimate the plurality of landmarks by applying medical images for the plurality of spaces of the cervical disc of a surgical patient to be operated to the generated learning model; and a cervical artificial disc modeling part configured to model the cervical artificial disc of the surgical patient by using the estimated plurality of landmarks.

Wherein, it is characterized in that the learning model generation part comprises: a training medical image collection part configured to collect training medical images for the plurality of spaces for the cervical disc; a training data generation part configured to generate training data by adding the plurality of landmarks to the collected training medical images; and an artificial intelligence training part configured to generate the learning model by training the learning model with the generated training data.

Wherein, it is characterized in that the learning model is configured to comprise individually generating each of the plurality of landmarks or collectively generating all the plurality of landmarks at a time, and the estimating of the plurality of landmarks is configured to perform individually estimating each of the plurality of landmarks according to the learning model, or collectively estimating all the plurality of landmarks at a time.

Wherein, it is characterized in that the cervical artificial disc modeling part is configured to generate a cervical artificial disc model of the surgical patient to be operated, by generating an available space into which the cervical artificial disc of the surgical patient to be operated is to be inserted according to the estimated plurality of landmarks, apply a predefined standard template of a cervical artificial disc to the generated available space, and reflect shape information extracted from the medical image matched to upper and lower surfaces and edges of the standard template, while matching the plurality landmarks to the medical image.

Wherein, it is characterized in that the plurality of spaces for the cervical disc are composed of spaces occupied by the cervical disc consisting of the plurality of landmarks between corresponding upper and lower cervical vertebrae, and the landmarks are set to comprise a plurality of landmarks respectively at a lower end of a upper end unit cranial vertebrae in a direction of a skull, and a upper end of a lower end unit caudal vertebrae in a direction of a spine, at a specific joint of the medical image.

Wherein, it is characterized in that the landmarks are configured to be set to comprise: at a center and left and right sides of a front outer region of the lower end of the upper end unit cervical vertebra to the direction of the skull, a cranial anterior center, a cranial anterior right, and a cranial anterior left, respectively; at a center of the lower end of the upper end unit cervical vertebra to the direction of the skull, a cranial apex; at a center and left and right sides of a rear outer region of the lower end of the upper end unit cervical vertebra to the direction of the skull, a cranial posterior center, a cranial posterior right, and a cranial posterior left, respectively; at a center and left and right sides of a front outer region of a upper end of a lower end unit caudal vertebra to a direction of a spine, a caudal anterior center, a caudal anterior near right, a caudal anterior far right, a caudal anterior near left, and a caudal anterior far left, respectively; and at a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.

Moreover, a method for modeling the artificial intelligence-based cervical artificial disc according to another embodiment of the present invention is characterized in that the method comprises: generating a learning model by training the learning model with a plurality of landmarks constituting a plurality of spaces of a cervical disc; estimating the plurality of landmarks by applying medical images for the plurality of spaces of the cervical disc of a surgical patient to be operated to the generated learning model; and modeling a cervical artificial disc of the surgical patient by using the estimated plurality of landmarks.

Wherein, it is characterized in that the generating of the learning model comprises collecting training medical images for the plurality of spaces of the cervical disc; generating training data by adding the plurality of landmarks to the collected training medical images; and generating the learning model by training the learning model with the generated training data.

Wherein, it is characterized in that the modeling of the cervical artificial disc comprises: generating a cervical artificial disc model of the surgical patient to be operated, by generating an available space into which the cervical artificial disc of the surgical patient to be operated is to be inserted according to the estimated plurality of landmarks; applying a predefined standard template of a cervical artificial disc to the generated available space; and reflecting shape information extracted from the medical image matched to upper and lower surfaces and edges of the standard template, while matching the plurality landmarks to the medical image.

As described above, according to the artificial intelligence-based cervical disc modeling device and method of the present invention, a learning model for estimating a plurality of landmarks is generated, coordinates of the plurality of landmarks are estimated by inputting a medical image of a user into the learning model for estimating the plurality of landmarks, and a cervical artificial disc of a user is generated using each of the coordinates of the estimated plurality of landmarks. Wherein, the artificial disc of the patient to be operated is modeled by adjusting the standard template to a size to fit an available space in which a corresponding cervical disc of a patient to be operated is located using the estimated plurality of landmarks, and matching the plurality of landmarks to a medical image of a patient to be operated to reflect shape information extracted from the matched medical image to upper and lower surfaces and edges of the template. By doing so, the present invention enables the cervical artificial disc to be manufactured in a customized manner by optimizing the cervical artificial disc on the surgical site of the user, and thus has an effect of improving the user satisfaction as well as a successful procedure through the custom-made cervical artificial disc.

### BRIEF DESCRIPTION OF THE DRAWINGS

FIG. 1 is a conceptual diagram schematically illustrating a usage environment of a device for modeling cervical artificial disc based on artificial intelligence according to an embodiment of the present disclosure.

FIG. 2 is a diagram for describing a process of generating an artificial intelligence learning model for a cervical artificial disc, estimating a plurality of landmarks through the artificial intelligence learning model, and generating a customized cervical artificial disc model according to an embodiment of the present disclosure.

FIG. 3 is a diagram for describing in a detail a process of generating a learning model for estimating a plurality of landmarks according to an embodiment of the present disclosure.

FIG. 4 is a diagram illustrating a plurality of landmarks set in a training medical image and A-space which is an available space represented by the plurality of landmarks in order to generate an artificial intelligence model according to an embodiment of the present disclosure.

FIG. 5 is a diagram for explaining each position of a plurality of landmarks in more detail according to an embodiment of the present disclosure.

FIG. 6 is a block diagram illustrating a configuration of a cervical artificial disc modeling device according to an embodiment of the present disclosure.

FIG. 7 is a conceptual diagram illustrating a process of performing a spatial estimation through a landmark estimation, applying the result of the spatial estimation to a template of a cervical artificial disc model, and generating a cervical artificial disc model according to an embodiment of the present disclosure.

FIG. 8 is a flowchart illustrating a method for modeling cervical artificial disc based on artificial intelligence according to an embodiment of the present disclosure.

FIG. 9 is a flowchart illustrating, in a detail, a process of generating a model of a customized cervical artificial disc from a medical image of a surgical patient of a cervical artificial disc according to an embodiment of the present disclosure.

### DETAILED DESCRIPTION OF THE EXEMPLARY EMBODIMENTS

Hereinafter, exemplary embodiments of the artificial intelligence-based cervical disc modeling device and method of the present invention are described in detail with reference to the accompanying drawings. The identical reference numerals indicated on each of the drawings denote the same components. In addition, specific structural or functional descriptions for embodiments of the present invention are only exemplified for the purpose of describing embodiments according to the present invention, and unless otherwise defined, all terms used herein, including technical or scientific terms, have the same meanings as commonly understood by one of ordinary skill in the art to which the present disclosure pertains. Commonly used terms, such as those defined in a dictionary should be interpreted to have a meaning that is consistent with their meaning in the context of the relevant art, and it is desirable that the terms are not to be interpreted in an idealized or overly formal sense unless explicitly defined herein in the specification.

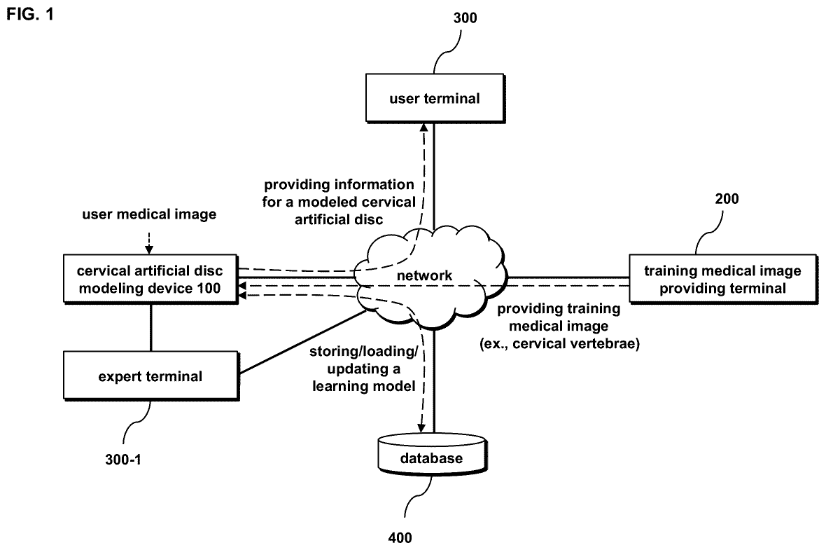

FIG. 1 is a conceptual diagram schematically illustrating a usage environment of a device for modeling cervical artificial disc based on artificial intelligence according to an embodiment of the present disclosure.

As shown in FIG. 1, the cervical artificial disc modeling device 100 of the present invention may be operated in a usage environment comprising a plurality of training medical images providing terminals 200, a user terminal 300, a database 400. In this case, an expert terminal 300-1 of a clinician having an input/output device (e.g., a monitor, a mouse, a keyboard, a camera, a 3D printer, a communication interface with medical equipment, etc.) can be further comprised so as to be directly connected to the cervical artificial disc modeling device 100 or connected to a network to control the cervical artificial disc modeling device 100.

The cervical artificial disc modeling device 100 collects the medical image of the cervical spine from the training medical image providing terminal 200 through a network, generates training data by determining a plurality of landmarks in the collected medical image, generates a learning model by performing training based on the generated training data, and stores the generated learning model in the database 400.

That is, the cervical artificial disc modeling device 100 generates training data by labeling each of a plurality of landmarks determined by an expert (e.g., a doctor, an image reading expert, an engineer, etc.) confirming the collected medical image, generates a learning model for estimating the plurality of landmarks by training the generated training data, and stores and manages the generated learning model in the database 400.

In this case, the plurality of landmarks are set (for example, 7 landmarks are set on the lower end of upper end unit cervical vertebra in the skull direction and 10 landmarks are set on the upper end of the lower end unit cervical vertebrae in the spine direction) on each of lower end of upper end unit cranial vertebrae in the skull direction and upper end of lower end unit caudal vertebrae in the spine direction in a specific joint (two unit cervical vertebrae and a disc there between) of a medical image.

The medical image is a medical image of users who have agreed to use personal information, has no risk of exposure of personal information, and includes 3D CT, MRI, and the like.

Wherein, when generating a learning model for estimating the plurality of landmarks, the cervical artificial disc modeling device 100 may generate a learning model based on a supervised learning, but may generate a learning model by using various learning methods including an unsupervised learning or a reinforcement learning.

Meanwhile, the cervical artificial disc modeling device 100 may pre-process a medical image of a user (patient) requiring cervical artificial disc replacement into a data format suitable for the landmark estimation learning model, may estimate coordinates for a plurality of landmarks individually or at once by inputting the pre-processed data format to the landmark estimation learning model, and may model the cervical artificial disc of the corresponding user by using each coordinate of the estimated plurality of landmarks.

The training medical image providing terminal 200 may be a terminal having a communication interface capable of providing the cervical vertebrae related image through a network in each medical institutions or data centers capturing a cervical vertebrae related medical image or managing a captured medical image.

Wherein, the medical image may be collected from each individual through PHR (personal health record), may be collected from each of a data center or a medical institution, and may be provided to the cervical artificial disc modeling device 100 according to a predetermined period or a request from a specific medical institution or a data center through a contract.

The user terminal 300 may be a communication terminal such as a smartphone, a tablet, a PC, etc. used by a user to whom cervical artificial disc replacement and the like are to be operated, and may obtain and verify information about the cervical artificial disc optimized for the user himself.

Furthermore, the expert terminal 300-1 has functions of allowing an expert to directly or through a network to operate, manage and control the cervical artificial disc modeling device 100 according to the present invention, beyond the functions of the user terminal.

That is, the expert terminal 300-1 and the user terminal 300 may accurately check the specification information including the height, length, width, shape, or a combination thereof with respect to the cervical artificial disc to be inserted into a surgical part by using the pre-installed application program.

The database 400 may store and manage a landmark estimation learning model generated by the cervical artificial disc modeling device 100, at least one standard template, and the like, and moreover may store and manage cervical vertebrae related medical images, patient IDs, patient passwords, and member information of each user (patient) in order to manufacture a customized cervical artificial disc.

Furthermore, the database 400 stores and manages a landmark estimation learning model used in the cervical artificial disc modeling device 100, landmarks estimated through the landmark estimation learning model, a model generation result of the cervical artificial disc, and information of the modeled cervical artificial disc. In addition, the database 400 may also include an application program for storing and managing the data, besides storing and managing various data listed above.

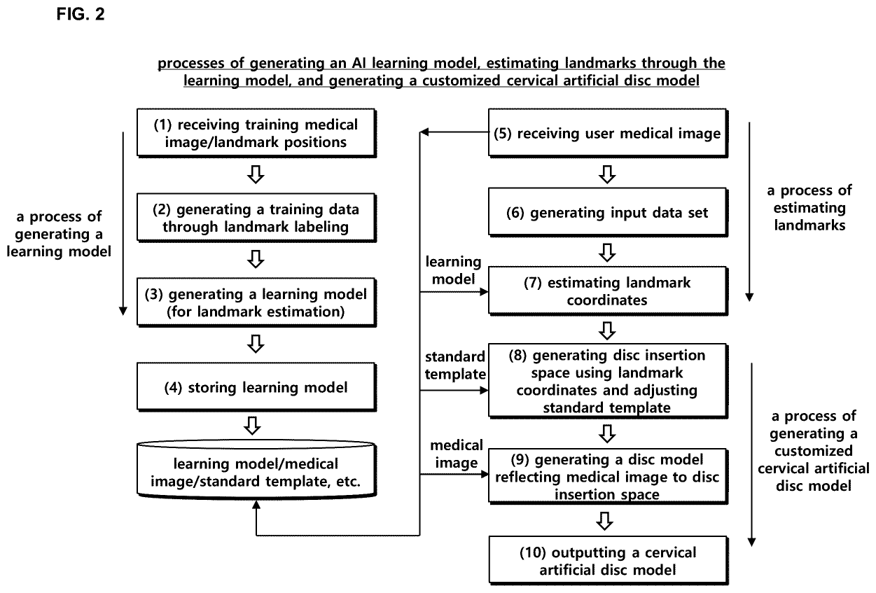

Next, a process of generating an artificial intelligence learning model, and estimating landmarks using the artificial intelligence learning model, and modeling a customized cervical artificial disc will be described in detail with reference to FIG. 2.

FIG. 2 is a diagram for describing a process of generating an artificial intelligence learning model for a cervical artificial disc, estimating a plurality of landmarks through the artificial intelligence learning model, and generating a customized cervical artificial disc model according to an embodiment of the present disclosure.

As shown in FIG. 2, the method according to the present invention comprises a process of generating an artificial intelligence learning model for a cervical artificial disc, a process of estimating landmarks through the generated artificial intelligence learning model, and a process of modeling for generating a customized cervical artificial disc model using the estimated landmarks.

First, a process of generating a learning model is described. The cervical artificial disc modeling device 100 collects a training medical image by scanning the cervical portion from the training medical image providing terminal 200 through a network. The landmarks of the training medical image determined by an expert can be further included along with the training medical image (1).

Subsequently, if a plurality of landmarks are received in each of the collected medical images, the received plurality of landmarks are used, if not, the plurality of landmarks are determined, the plurality of landmarks are labeled, and then the training data is generated (2), the generated training data is input to train the landmark estimation learning network (3), and the learning model generated through the training is stored in a database (4). That is, the cervical artificial disc modeling device 100 stores and manages the generated landmark estimation learning model in the database 400. Through the above processes, a learning model is generated and stored and managed.

Hereinafter, a process of estimating a plurality of landmarks is described. The cervical artificial disc modeling device 100 receives a medical image of a user from the database 400 or a medical device (for example, a medical device such as CT, MRI, etc.), in order to model a cervical artificial disc suitable for the user (patient) undergoing a cervical disc surgery (5). The received medical image of the user is converted into a data format (a data set representing an available space into which an artificial disc is to be inserted) used in the landmark estimation learning model, and thus generating an input data set (6). The generated input data set (i.e., medical image of a user) is input to the landmark estimation learning model to estimate coordinates of the plurality of landmarks for the disc surgery part (7). Thereby, completing the process of estimating the plurality of landmarks.

Next, a process of generating a customized cervical artificial disc model is described hereinafter. The cervical artificial disc modeling device 100 generates a model of the cervical artificial disc by using each of coordinates of the plurality of landmarks estimated from the medical image of the user. To this end, first generating the available space in which the disc is to be inserted using the coordinates of the plurality of landmarks estimated, then applying the available space to the previously stored standard template and adjusting the size of the standard template (8). The shape appearing in the cervical vertebrae located on the upper and lower sides of the disc to be operated or surrounding image of the disc is reflected in the modified template having the adjusted size (9). That is, three-dimensional coordinates for each of the detailed feature points from the shape observed in medical images captured (scanned) of an available space between the upper and lower cervical vertebrae of the surgical patient are extracted and the three-dimensional coordinates for each of the detailed feature points are reflected on the surface of the adjusted template (9), and the template is further modified to generate a cervical artificial disc model and the results of the generated cervical artificial disc model are outputted (10). The cervical artificial disc modeling device 100 may generate information including a height, a length, an area, a shape, or a combination thereof with respect to the modeled specific cervical artificial disc template, as an image, text, or a combination thereof, may provide the generated information to the user terminal 300 or the expert terminal 300-1, and may output the generated information to a device or a tool for manufacturing a cervical artificial disc such as a 3D printer (10). Accordingly, a process of generating a patient customized cervical artificial disc model according to the present invention has been described.

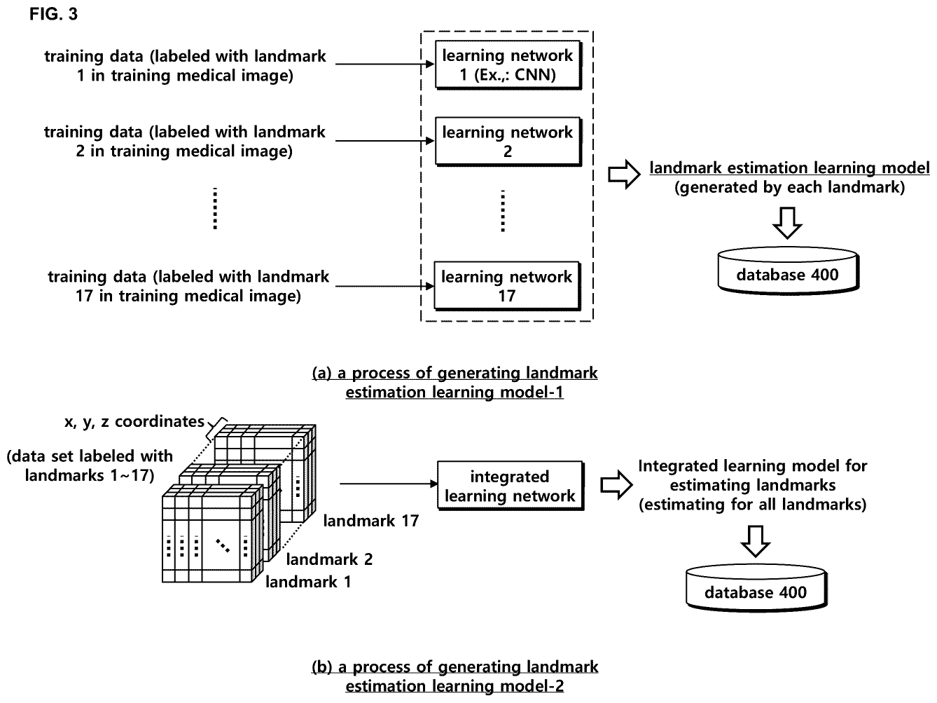

Hereinafter, a process of generating the landmark estimation learning model is described in detail with reference to FIG. 3.

FIG. 3 is a diagram for describing in a detail a process of generating a learning model for estimating a plurality of landmarks according to an embodiment of the present disclosure.

First, as shown in (a) of FIG. 3, the cervical artificial disc modeling device 100 trains a learning network (model) by inputting a medical image for each user labeled with landmarks (i.e., 1), generates a landmark (i.e., 1) estimating learning model by deriving an optimal parameter set of the learning network, and stores the generated landmark estimation learning model in the database 400.

At this time, the medical image used for the training may use a two-dimensional X-ray image, but it is preferable to use a three-dimensional medical image such as CT or MRI, which is capable of three-dimensionally identifying the cervical vertebrae part from six different perspectives.

In addition, the cervical artificial disc modeling device 100 generates a landmark (for 2 to 17) estimating learning model by performing training using a medical image for each user, in which landmarks 2 to 17 are set, respectively, in the same manner as when generating the landmark (i.e., 1) estimating learning model, and stores the generated landmark (2 to 17) estimating learning model in the database 400.

In addition, each landmark estimation learning model for each of landmarks according to the present invention is configured to simultaneously perform a plurality of training through a multi-task. That is, the plurality of learning networks are trained in parallel, and the results are stored as a learning model.

Meanwhile, as shown in (b) of FIG. 3, the training data for each of the plurality of landmarks is configured by registering three-dimensionally combined training data, thereby configuring a learning model to simultaneously estimate a plurality of landmarks at a time.

The training data may be configured to a training data in which a plurality of landmarks are integrated by adding one dimension to an individual data set for a plurality of landmarks, and an integrated learning network using the configured training data as an input may be configured, and an integrated learning model can be generated by training the learning network with the training data.

For example, the integrated learning model may be generated by labeling the three-dimensional coordinates for each of the landmarks, making one data set with the coordinates, and inputting the data set to an integrated learning network. In this case, since each of the landmarks is labeled individually, and thereby there is no relation among the landmarks, it may be desirable to have an independent learning network structured for each landmark. That is, the integrated learning network may be configured as a network having a three-dimensional shape, such as a shape of input training data. In addition, 17 output data sets are simultaneously output and stored in the database 400.

In addition, the cervical artificial disc modeling device 100 generates an integrated learning model using, for example, a three-dimensional CNN according to training data generated in the three-dimensional structure, and stores and manages the generated integrated learning model in the database 400.

In order to generate a landmark estimation learning model, a convolution neural network (CNN), as a learning network, may be used for training the landmark estimation learning model, and the CNN comprises an input layer to which training data is input, a convolution layer, a pooling layer, and a fully connected layer.

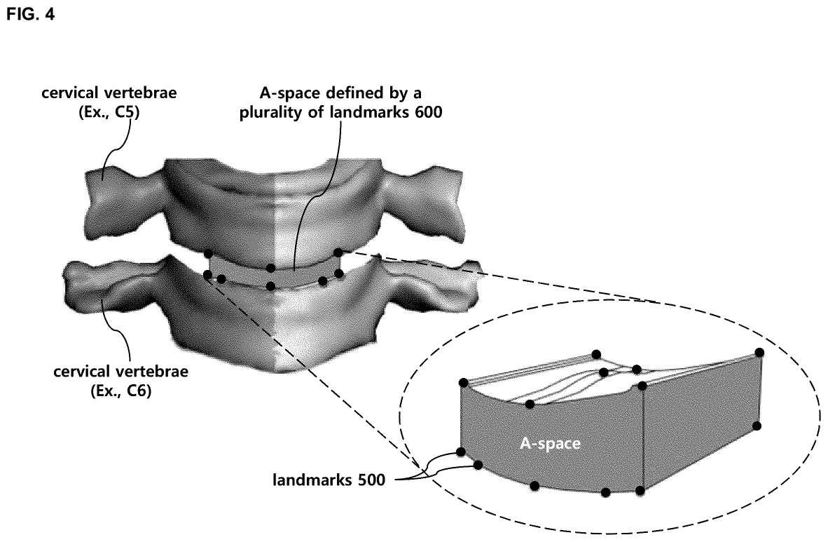

FIG. 4 is a diagram illustrating a plurality of landmarks set in a training medical image and A-space which is an available space represented by the plurality of landmarks in order to generate an artificial intelligence model according to an embodiment of the present disclosure.

As shown in FIG. 4, the A-space 600 is a space defined by a plurality of landmarks 500, and models a space between two upper and lower cervical vertebrae (for example, between the C5 cervical vertebrae and the C6 cervical vertebrae) at a position where the cervical artificial disc is to be operated.

In other words, when designing a single cervical artificial disc, the A-space 600 refers to a space defined as a set of feature points (landmarks) representing available size and shape in which the cervical artificial disc is inserted between the upper and lower cervical vertebrae of a place where the cervical artificial disc is operated.

Furthermore, since the A-space 600 is modeled differently depending on the cervical condition or the surgical site of the user to be operated, the cervical artificial disc most suitable for the user to be operated is modeled from the A-space 600 modeled according to the method of the present invention, thereby resolving the problem of decreased surgical success probability that occurs due to the surgery using a ready-made cervical artificial disc designed according to a predetermined specification in traditional methods.

The A-space, which is an available space in which a specific cervical artificial disc is to be located, is defined by experts who assign landmarks to the upper, lower, left, and right sides of corresponding medical image for the specific cervical artificial disc. While the landmarks can characterize the respective space, there still exist undefined areas between each of landmarks.

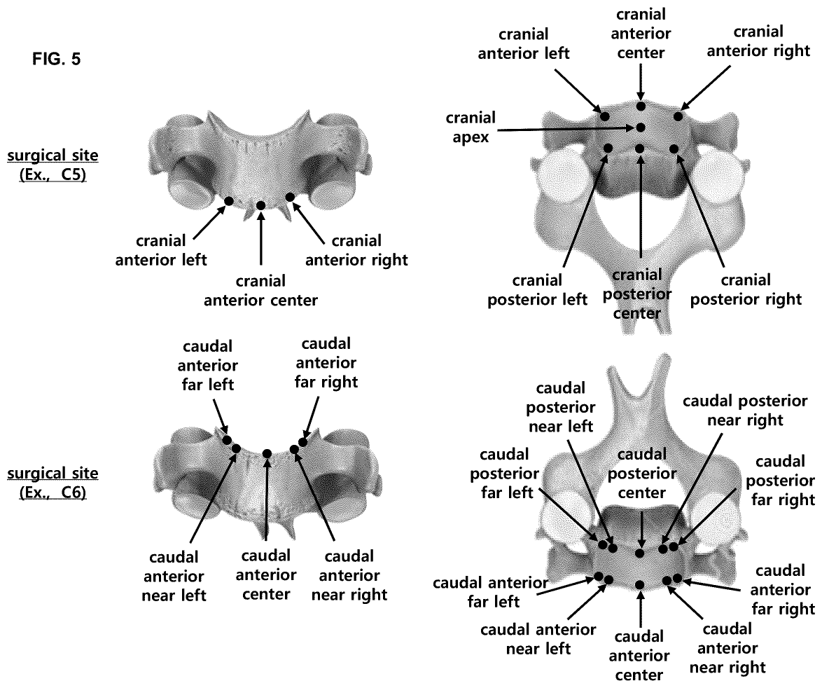

FIG. 5 is a diagram for explaining each position of a plurality of landmarks in more detail according to an embodiment of the present disclosure.

As shown in FIG. 5, a plurality of landmarks 500 are set to ensure coverage sufficiently surrounding a unit cervical vertebra of a surgical site, and set to guide the unit cervical vertebra to be implanted aligning with a central line, and A-space 600, which is a space into which the cervical artificial disc is to be inserted by using the plurality of landmarks 500 is three-dimensionally modeled.

For example, for the landmarks 500, 7 landmarks are set at the lower end of the upper end unit cranial vertebrae to the direction of skull and 10 landmarks are set at the upper end of lower unit caudal vertebrae to the direction of spine, at a specific joint.

In other words, the landmarks 500 are configured to set to comprise, at the center and the left and right sides of the front outer region of the lower end of the upper end unit cervical vertebra to the direction of skull, a cranial anterior center, a cranial anterior right, and a cranial anterior left, respectively, at the center of the lower end of the upper end unit cervical vertebra to the direction of the skull, a cranial apex, and at the center and left and right sides of rear outer region of the lower end of the upper end unit cervical vertebra to the direction of the skull, a cranial posterior center, a cranial posterior right, and a cranial posterior left, respectively.

In addition, the landmarks 500 are configured to set to comprise, at the center and left and right sides of front outer region of the upper end of the lower end unit cervical vertebra to the direction of the spine, a caudal anterior center, a caudal anterior near right, a caudal anterior far right, a caudal anterior near left, and a caudal anterior far left, respectively, and at the center and left and right sides of rear outer region of the upper end of the lower end unit cervical vertebra to the direction of the spine, a caudal posterior center, a caudal posterior near right, a caudal posterior far right, a caudal posterior near left, and a caudal posterior far left, respectively.

Wherein, in the present invention, although a total 17 of the landmarks 500 are set as an example, but the present invention is not limited thereto, and it is clearly stated that the number of landmarks may be increased or decreased for use.

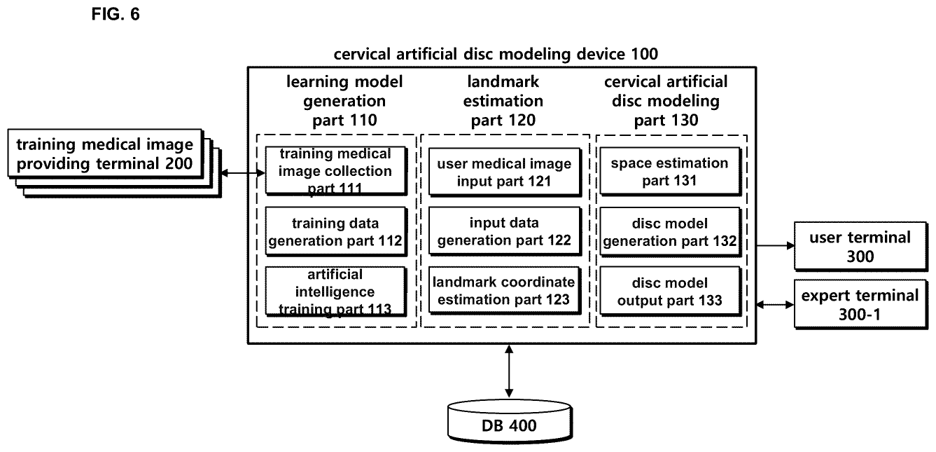

FIG. 6 is a block diagram illustrating a configuration of a cervical artificial disc modeling device according to an embodiment of the present disclosure.

As shown in FIG. 6, the cervical artificial disc modeling device 100 comprises a learning model generation part 110, a landmark estimation part 120, and a cervical artificial disc modeling part 130.

The learning model generation part 110 comprises a training medical image collection part 111, a training data generation part 112, and an artificial intelligence training part 113.

Although not shown in the drawings, the cervical artificial disc modeling device 100 further comprises, as a hardware point of view, a processor, a memory, a bus connecting the processor and the memory, and various interface cards, and further comprises, as a software point of view, programs stored in the memory and driven through the processor, a user interface performing operations according to commands from a user or the network, an update management part for managing updates of various operational programs, and an interface part for transmitting and receiving data to/from an external device such as a database.

The training medical image collection part 111 is configured to collect training medical images for a plurality of cervical disc spaces from a learning medical image providing terminal 200 connected to a network or directly connected to the learning medical image providing terminal 200. In this case, each medical image may include landmarks designated by an expert.

The collected training medical image may not include a landmark, and thus a task of determining individual landmarks may be performed in the cervical artificial disc modeling device 100 according to the present invention.

In this case, the medical image may be collected from each individual through PHR (personal health record), and may be collected from a data center or each medical institution, and may be provided in real time according to a predetermined period or request from a specific medical institution or data center through a contract.

The training data generation part 112 is configured to generate training data by adding a plurality of landmarks to the collected training medical image. The training data is provided by a preprocessing process for inputting training data to an artificial intelligence learning network to perform training, and may have a one-dimensional, two-dimensional, three-dimensional, or a plurality of shapes in which they are coupled. The generated training data is labeled according to each landmark.

In addition, the artificial intelligence training part 113 is configured to generate an artificial intelligence learning model for landmark estimation by extracting training parameters of the learning network by training the learning network by inputting the generated training data to the learning network.

That is, the artificial intelligence learning part 113 is configured to generate training data by determining a plurality of landmarks in each medical image collected by the training medical image collection part 111, generate a landmark estimation learning model trained with the generated training data, and store and manage the generated landmark estimation learning model in the database 400.

Wherein, a main function of the learning model generation part 120 is to determine a plurality of landmarks, perform labeling for the determined plurality of landmarks, and train an artificial intelligence learning network using the labeled plurality of landmarks.

Wherein, the determining of the plurality of landmarks refers to determining three-dimensional coordinates for each of a plurality of landmarks in each of the collected medical images, and the labels is used for distinguishing each of the landmarks by assigning labels to the plurality of determined landmarks, and the artificial intelligence training is to generate a landmark estimation learning model by performing training based on the determined landmarks and each medical image labeled with the landmarks.

Hereinafter, the landmark estimation part 120 is described in detail. The landmark estimation part 120 comprises a user medical image input part 121, an input data generation part 122, and a landmark coordinate estimation part 123.

The user medical image input part 121 is configured to receive medical images of the upper and lower cervical vertebrae of a surgical target cervical disc of a surgical patient from the database 400 or medical devices. The received medical image is output to the input data generation part 122.

The input data generation part 122 is configured to perform preprocessing to convert the medical image into a data format for applying the medical image to a learning model.

Subsequently, the landmark coordinate estimation part 123 is configured to estimate a plurality of landmark coordinates, by inputting the input data to the landmark estimation learning model generated by the training model generation part 110.

Information on each coordinate of the estimated landmarks is output to the cervical artificial disc modeling part 130. In this case, when estimating a plurality of landmark coordinates through the landmark estimation learning model, the landmark estimation part 120 is configured to estimate each landmark or integrally estimate the landmarks at once.

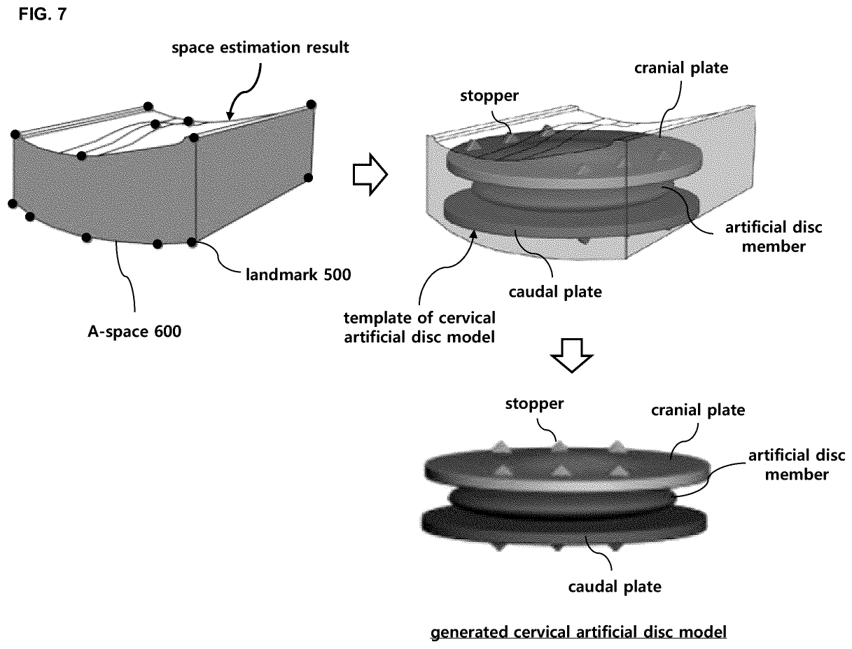

Hereinafter, the cervical artificial disc modeling part 130 is described with reference to FIG. 7. FIG. 7 is a conceptual diagram illustrating a process of performing a spatial estimation through a landmark estimation, applying the result of the spatial estimation to a template of a cervical artificial disc model, and generating a cervical artificial disc model according to an embodiment of the present disclosure.

That is, an available space is generated by the landmarks estimated by the landmark estimation part 120, and a standard template is inserted in the generated available space, and the size of the template is fit into the available space. Wherein, the fixing stopper provided at the upper and lower sides of the cervical artificial disc is configured to be fixed to the upper and lower cervical vertebrae beyond the estimated range.

The cervical artificial disc modeling part 130 is configured to comprise a space estimation part 131 configuring a space of a disc model by using the estimated landmarks, a disc model generation part 132 configured to generate a disc model by supplementing the configured disc space with an actual medical image, and a disc model output part 133 configured to output the generated disc model.

The space estimation part 131 is configured to estimate the space of the cervical artificial disc to be operated by using each of coordinates of the landmarks estimated from the medical image of the surgical patient through the landmark estimation part 120. Wherein, the estimation of the disc space refers to the process of estimating and creating the space in which the disc is to be located between the upper and lower cervical vertebrae of the cervical disc to be operated by connecting the plurality of landmarks each other. The created space is the same as a peripheral housing into which an actual artificial disc is to be inserted.

The disc model generating part 132 is configured to generate an artificial disc model to be inserted to be fit into the created space. In this process, a standard template for the artificial disc model is retrieved from a memory or a database, inserted into the estimated space, and the size of the standard template is fit into the estimated space. The surface and the edge of the fitted template are not adequately modeled only by using the standard template. Therefore, the shapes of upper and lower surfaces and peripheral edges of the template are retrieved and reflected from medical images of a patient.

The created template thus becomes the cervical artificial disc model.

The disc model output part 133 is configured to transmit the created cervical artificial disc model to an output device such as an external 3D printer to make a final product. In addition, before finally outputting the final product to the output device, it is possible to output the created model to the user terminal 300 or the expert terminal 300-1. That is, it is possible to manufacture a customized cervical artificial disc through modeling of the cervical artificial disc that is most suitable for the user.

The disc model output part 133 is configured to generate result data comprising image, text, or a combination thereof on the basis of information including a height, a length, an area, a shape, or a combination thereof for the cervical artificial disc created by the disc model creating part 132 in addition to outputting the actually created disc model to 3D printer, etc., and provide the generated result data to an intended surgical user or an expert.

In addition, the cervical artificial disc modeling device 100 is configured to comprise an individual memory (not shown) therein. Wherein the individual memory is configured to store various operation programs used in the cervical artificial disc modeling device 100, and temporarily store result data for each of medical images collected from the training medical image providing terminal 200, a landmark estimation result through the landmark estimation part 120, and result data for a customized cervical artificial disc modeled through the cervical artificial disc modeling part 130.

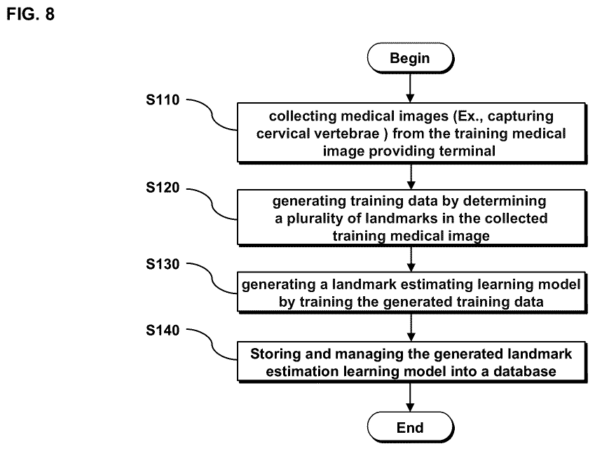

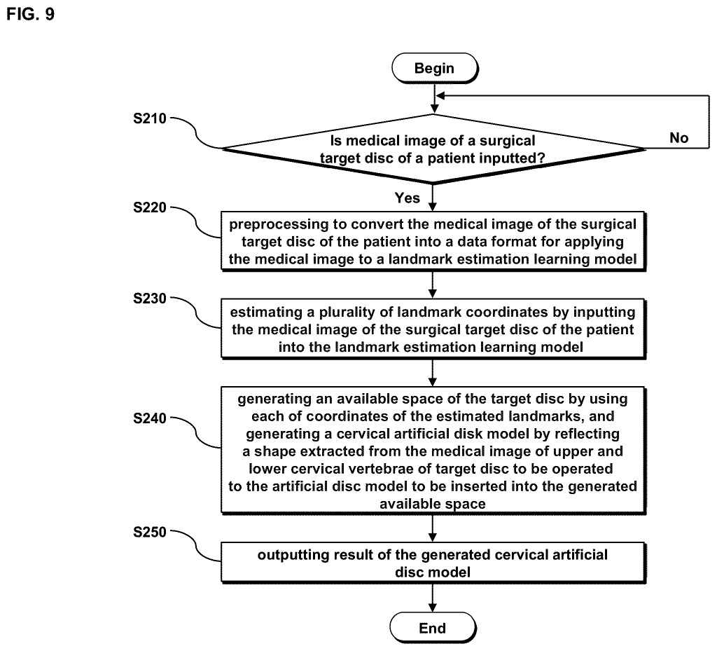

Hereinafter, an embodiment of a method for the artificial intelligence-based cervical disc modeling configured as described above according to the present invention is described in detail with reference to FIG. 8 and FIG. 9. Herein, it should be noted that the order of each process according to the method of the present invention may be changed by a usage environment or a person skilled in the art.

FIG. 8 is a flowchart illustrating an artificial intelligence-based cervical disc modeling method according to an embodiment of the present disclosure.

As shown in FIG. 8, the cervical artificial disc modeling device 100 is configured to perform a training medical image collection process of collecting each of medical images from the training medical image providing terminal 200, S110.

Subsequently, the cervical artificial disc modeling device 100 is configured to perform a process of generating training data by determining a plurality of landmarks in the training medical image collected from the training medical image providing terminal 200 through the process of S110, S120, and perform a learning model generation process of generating a landmark estimation learning model by training the generated training data, S130.

That is, the cervical artificial disc modeling device 100 is configured to generate a landmark estimation learning model by training medical images for each user for the cervical part collected from the training medical image providing terminal 200 and each of medical images labeled with a plurality of landmarks determined by the expert who has examined the medical image.

In addition, the cervical artificial disc modeling device 100 is configured to store and manage the landmark estimation learning model generated through the S130 in the database 400, S140.

Meanwhile, the cervical artificial disc modeling device 100 is configured to generate a landmark estimation learning model and then model the customized cervical artificial disc from the medical image of the user, which is described in detail with reference to FIG. 9.

FIG. 9 is a flowchart illustrating, in a detail, a process of generating a model of a customized cervical artificial disc from a medical image of a surgical patient of a cervical artificial disc according to an embodiment of the present disclosure.

As shown in FIG. 9, the cervical artificial disc modeling device 100 is configured to determine whether a medical image (that is, a medical image such as CT, MRI, etc. capturing the cervical region) of a user who is intended for cervical artificial disc replacement surgery is input, S210. When the medical image of the user is input as a result of the determination in S210, the cervical artificial disc modeling device 100 is configured to perform preprocessing to convert the medical image of the user into a data format for applying the medical image of the user to the landmark estimation learning model generated in the S130 or the S150, S220.

After preprocessing the medical image of the user through the S220, the cervical artificial disc modeling device 100 is configured to perform a landmark coordinate estimation process of estimating a plurality of landmark coordinates by inputting the medical image of the user into a landmark estimation learning model generated through the S130, S230.

Subsequently, the cervical artificial disk modeling apparatus 100 is configured to model the cervical artificial disc of the user through each of coordinates of the landmarks estimated through the S230, generate an available space of the target disc by using each of coordinates of the estimated landmarks, and generate a cervical artificial disk model by reflecting a shape extracted from the medical image of upper and lower cervical vertebrae of target disc to be operated to the artificial disc model to be inserted into the generated available space, S240.

Accordingly, it is possible to manufacture the cervical artificial disc most suitable for the surgical site of the user in a customized manner through the information on the cervical artificial disc modeled through the S240.

Furthermore, the cervical artificial disc modeling device 100 is configured to generate, as image, text or a combination thereof, information including a height, a length, an area, a shape, or a combination thereof for the cervical artificial disc modeled through the S240, and perform a result output process of providing the information to a user or an expert, S250.

Accordingly, according to the present invention, a learning model for estimating positions for the plurality of landmarks is generated by training medical images of various shapes to which a plurality of landmarks constituting an available space in which the cervical disc is located are assigned, the plurality of landmarks are estimated by inputting input data generated from the medical image of the available space in which the surgical target cervical artificial disc is positioned to the generated learning model, the available space into which surgical target cervical artificial disc is inserted is generated according to the estimated plurality of landmarks, a predefined standard template of a cervical artificial disc is applied to the generated available space (size adjustment, etc.), shape information extracted from the matched medical image is reflected to upper and lower surfaces and edges of the standard template in a state of matching the landmarks to the medical image, and thereby generating and outputting a surgical target cervical artificial disc model.

As described above, according to the artificial intelligence-based cervical disc modeling device and method of the present invention, a learning model for estimating a plurality of landmarks is generated, coordinates of the plurality of landmarks are estimated by inputting a medical image of a user into the learning model for estimating the plurality of landmarks, and a cervical artificial disc of a user is generated using each of the coordinates of the estimated plurality of landmarks. Wherein, the artificial disc of the patient to be operated is modeled by adjusting the standard template to a size to fit an available space in which a corresponding cervical disc of a patient to be operated is located using the estimated plurality of landmarks, and matching the plurality of landmarks to a medical image of a patient to be operated to reflect shape information extracted from the matched medical image to upper and lower surfaces and edges of the template. By doing so, the present invention enables the cervical artificial disc to be manufactured in a customized manner by optimizing the cervical artificial disc on the surgical site of the user, and thus has an effect of improving the user satisfaction as well as a successful procedure through the custom-made cervical artificial disc.

As described above, embodiments according to the present invention have been described with reference to the drawings, but these are merely examples, and it will be understood by a person skilled in the art that various modifications and other equivalent embodiments are possible therefrom. Therefore, the technical scope of the present invention should be determined by the following claims.

The artificial intelligence-based cervical artificial disc modeling device and method according to the present invention can be realized by manufacturing a cervical artificial disc in a customized manner by optimizing the cervical artificial disc to a surgical site of a user, and thereby promising an improvement of user satisfaction as well as a successful surgical procedure through the artificial cervical disc manufactured in customized manner.

What is claimed is:

1. A device for modeling a cervical artificial disc based on artificial intelligence, the device comprising:
a learning model generation part configured to generate a learning model through training a plurality of landmarks constituting a plurality of spaces for a cervical disc;
a landmark estimation part configured to estimate the plurality of landmarks by applying medical images for the plurality of spaces of the cervical disc of a surgical patient to be operated to the generated learning model; and
a cervical artificial disc modeling part configured to model the cervical artificial disc of the surgical patient by using the estimated plurality of landmarks.

2. The device of claim 1, wherein the learning model generation part comprises:
a training medical image collection part configured to collect training medical images for the plurality of spaces for the cervical disc;
a training data generation part configured to generate training data by adding the plurality of landmarks to the collected training medical images; and
an artificial intelligence training part configured to generate the learning model by training the learning model with the generated training data.

3. The device of claim 1, wherein the learning model is configured to comprise individually generating each of the plurality of landmarks or collectively generating all the plurality of landmarks at a time; and
wherein the estimating of the plurality of landmarks is configured to perform individually estimating each of the plurality of landmarks according to the learning model, or collectively estimating all the plurality of landmarks at a time.

4. The device of claim 1, wherein the cervical artificial disc modeling part is configured to generate a cervical artificial disc model of the surgical patient to be operated, by generating an available space into which the cervical artificial disc of the surgical patient to be operated is to be inserted according to the estimated plurality of landmarks, apply a predefined standard template of a cervical artificial disc to the generated available space, and reflect shape information extracted from the medical image matched to upper and lower surfaces and edges of the standard template, while matching the plurality landmarks to the medical image.

5. The device of claim 1, wherein the plurality of spaces for the cervical disc are composed of spaces occupied by the cervical disc consisting of the plurality of landmarks between corresponding upper and lower cervical vertebrae; and
wherein the landmarks are set to comprise a plurality of landmarks respectively at a lower end of a upper end unit cranial vertebrae in a direction of a skull, and a upper end of a lower end unit caudal vertebrae in a direction of a spine, at a specific joint of the medical image.

6. The device of claim 5, wherein the landmarks are configured to be set to comprise:
at a center and left and right sides of a front outer region of the lower end of the upper end unit cervical vertebra to the direction of the skull, a cranial anterior center, a cranial anterior right, and a cranial anterior left, respectively,
at a center of the lower end of the upper end unit cervical vertebra to the direction of the skull, a cranial apex,
at a center and left and right sides of a rear outer region of the lower end of the upper end unit cervical vertebra to the direction of the skull, a cranial posterior center, a cranial posterior right, and a cranial posterior left, respectively,
at a center and left and right sides of a front outer region of a upper end of a lower end unit caudal vertebra to a direction of a spine, a caudal anterior center, a caudal anterior near right, a caudal anterior far right, a caudal anterior near left, and a caudal anterior far left, respectively, and
at a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.

7. A method for modeling a cervical artificial disc based on artificial intelligence, the method comprising:
generating a learning model by training the learning model with a plurality of landmarks constituting a plurality of spaces of a cervical disc;
estimating the plurality of landmarks by applying medical images for the plurality of spaces of the cervical disc of a surgical patient to be operated to the generated learning model; and
modeling a cervical artificial disc of the surgical patient by using the estimated plurality of landmarks.

8. The method of claim 7, wherein the generating of the learning model comprises:
collecting training medical images for the plurality of spaces of the cervical disc;
generating training data by adding the plurality of landmarks to the collected training medical images; and
generating the learning model by training the learning model with the generated training data.

9. The method of claim 7, wherein the learning model is configured to comprise individually generating each of the plurality of landmarks or collectively generating all the plurality of landmarks at a time; and
wherein the estimating of the plurality of landmarks is configured to perform individually estimating each of the plurality of landmarks according to the learning model, or collectively estimating all the plurality of landmarks at a time.

10. The method of claim 7, wherein the modeling of the cervical artificial disc comprises:
generating a cervical artificial disc model of the surgical patient to be operated, by generating an available space into which the cervical artificial disc of the surgical patient to be operated is to be inserted according to the estimated plurality of landmarks;
applying a predefined standard template of a cervical artificial disc to the generated available space; and
reflecting shape information extracted from the medical image matched to upper and lower surfaces and edges of the standard template, while matching the plurality landmarks to the medical image.

11. The method of claim 7, wherein the plurality of spaces for the cervical disc are composed of spaces occupied by the cervical disc consisting of the plurality of landmarks between corresponding upper and lower cervical vertebrae; and
wherein the landmarks are set to comprise a plurality of landmarks respectively at a lower end of a upper end unit cranial vertebrae in a direction of a skull, and a upper end of a lower end unit caudal vertebrae in a direction of a spine, at a specific joint of the medical image.

12. The method of claim 11, wherein the landmarks are configured to be set to comprise:
at a center and left and right sides of a front outer region of the lower end of the upper end unit cervical vertebra to the direction of the skull, a cranial anterior center, a cranial anterior right, and a cranial anterior left, respectively,
at a center of the lower end of the upper end unit cervical vertebra to the direction of the skull, a cranial apex,
at a center and left and right sides of a rear outer region of the lower end of the upper end unit cervical vertebra to the direction of the skull, a cranial posterior center, a cranial posterior right, and a cranial posterior left, respectively,
at a center and left and right sides of a front outer region of a upper end of a lower end unit caudal vertebra to a direction of a spine, a caudal anterior center, a caudal anterior near right, a caudal anterior far right, a caudal anterior near left, and a caudal anterior far left, respectively, and
at a center and left and right sides of a rear outer region of the upper end of the lower end unit caudal vertebra to the direction of the spine, a caudal posterior center, a caudal posterior near right, a caudal posterior far right, a caudal posterior near left, and a caudal posterior far left, respectively.
